(19) United States
(12) Patent Application Publication
(10) Pub. No.: US 2011/0282400 A1
(43) Pub. Date: Nov. 17, 2011

(54) Reverse angled threadform with anti-splay Clearance

(76) Inventor: Roger P. Jackson, Prairie Village, KS (US)

(21) Appl. No.: 13/135,963

(22) Filed: Jul. 19, 2011

Related U.S. Application Data
(62) Division of application No. 11/285,094, filed on Nov. 22, 2005.
Application No. 11/285,094 is a continuation-in-part of application No. 09/644,777, filed on Aug. 23, 2000.
Application No. 09/644,777 is a continuation-in-part of application No. 11/246,320, filed on Oct. 7, 2005.

(51) Int. Cl. A61B 17/86

(52) U.S. Cl. 606/305

(57) ABSTRACT

A reverse angled threadform with anti-splay clearance includes inner and outer helical threads, each with respective leading stab flanks and trailing load flanks, the load flanks mutually engaging when an inner member with the inner threads is advanced into an outer member with the outer threads. The inner load flanks form acute angles with a helical axis, while the outer load flanks form obtuse angles with the helical axis. The clearance is formed between the stab flanks of the inner and outer threads. On threadforms with root and crest other than angular peaks, such as cylindrical root and crest surfaces, the anti-splay clearance is also formed between the mutually facing root and crest surfaces of the inner and outer threads. The load flanks may be parallel, outwardly diverging, or outwardly converging.

### CROSS-REFERENCE TO RELATED APPLICATION

This is a divisional of U.S. patent application Ser. No. 11/285,094 for REVERSE ANGLED THREADFORM WITH ANTI-SPLAY CLEARANCE, filed Nov. 22, 2005, which was a continuation-in-part of U.S. patent application Ser. No. 09/644,777 for THREADFORM FOR MEDICAL IMPLANT CLOSURE filed Aug. 23, 2000, now U.S. Pat. No. ______, and a continuation-in-part of U.S. patent application Ser. No. 11/246,320 for HELICAL REVERSE ANGLE GUIDE AND ADVANCEMENT STRUCTURE WITH BREAK-OFF EXTENSIONS, filed Oct. 7, 2005, now U.S. Pat. No. ______, which are incorporated herein by reference.

### BACKGROUND OF THE INVENTION

The present invention relates to improvements in helical guide and advancement structures such as threads and to forming guide and advancement structures in such a manner as to control the relative loading or stressing of the male and female components of such structures. More particularly, the present invention relates to forming reverse angled threads with parallel, diverging, or converging load and stab flanks in such a manner as to control relative loading of male and female components of such threads. Additionally, the threads of the present invention are configured to provide anti-splay clearance between portions of the threads to enable portions of the outer member incorporating such threads to be drawn toward the inner member.

Medical implants present a number of problems to both surgeons installing implants and to engineers designing them. It is always desirable to have an implant that is strong and unlikely to fail or break during usage. Further, if one of a set of cooperating components is likely to fail during an implant procedure, it is desirable to control which particular component fails and the manner in which it fails, to avoid injury and to minimize surgery to replace or repair the failed component. It is also desirable for the implant to be as small and lightweight as possible so that it is less intrusive to the patient. These are normally conflicting goals, and often difficult to resolve.

One type of implant presents special problems. In particular, spinal bone screws, hooks, and the like are used in many types of back surgery for repair of problems and deformities of the spine due to injury, disease or congenital defect. For example, spinal bone screws typically have one end that threads into a vertebra and a head at an opposite end. The head is formed with an opening to receive a rod or rod-like member which is then both captured in the channel and locked in the head to prevent relative movement between the various elements subsequent to installation.

A particularly useful type of head for such bone screws is an open head wherein an open, generally U-shaped channel is formed in the head, and the rod is simply laid in the open channel. The channel is then closed with some type of a closure member which engages the walls or arms forming the head and clamps the rod in place within the channel. While the open headed devices are often necessary and preferred for usage, there is a significant problem associated with them. The open headed devices conventionally have two upstanding arms that are on opposite sides of the channel that receives the rod member. The top of the channel is closed by a closure member after the rod member is placed in the channel. Many open headed implants are closed by closure plugs or closures that screw into threads formed on internal surfaces between the arms, because such configurations have low profiles.

However, such threaded closures have encountered problems in that they produce radially outward forces that lead to splaying of the arms or at least do not prevent splaying that in turn loosens the implant. In order to lock the rod-like member or longitudinal connecting member in place, a significant force must be exerted on the relatively small closure or screw. The forces are required to provide enough torque to insure that the connecting member is clamped or locked securely in place relative to the bone screw, so that this member does not move axially or rotationally therein. This typically requires torques on the order of 100 inch-pounds.

Because open headed implants such as bone screws, hooks and the like are relatively small, the arms that extend upwardly at the head can be spread by radially outwardly directed forces in response to the application of the substantial torquing force required to clamp the rod or rod-like member. Historically, early closures were simple plugs that were threaded with V-shaped threads and which screwed into mating threads on the inside of each of the arms. The outward flexure of the arms of the head is caused by mutual camming action of the V-shaped threads of the closure and head as advancement of the closure is resisted by clamping engagement with the rod while rotational urging of the closure continues. If the arms are sufficiently spread, they can allow the threads to loosen or disengage and the closure to fail. To counter this, various engineering techniques were applied to the head to increase its resistance to the spreading force. For example, the arms were strengthened by significantly increasing the width of the arms. Alternatively, external caps were devised which engaged external surfaces of the head. In either case, the unfortunate effect was to substantially increase the weight, size, and the profile of the implant.

The radial expansion problem of V-threads has been recognized in various other applications of threaded joints. To overcome this problem, so-called “buttress” threadforms were developed. In a buttress thread, the trailing or thrust surface, also known as the load flank, is oriented perpendicular to the thread axis, while the leading or clearance surface, also known as the stab flank, remains angled. This results in a neutral radial reaction of a threaded receptacle to torque on the threaded member received.

Development of threadforms proceeded from buttress threadforms and square threadforms, which have a neutral radial effect on the screw receptacle, to reverse angled threadforms which positively draw the threads of the receptacle radially inward toward the thread axis when the closure is torqued. In a reverse angle threadform, the trailing side of the external thread is angled toward the thread axis instead of away from the thread axis, as in conventional V-threads.

When rods are used in spinal fixation systems, it is often necessary to shape the rod in various ways to properly position vertebrae into which open headed bone screws have been implanted. The heads of bone screw heads are minimized in length to thereby minimize the impact of the implanted system on the patient. However, it is often difficult to capture a portion of a curved rod in a short bone screw head to clamp it within the bone screw.

### SUMMARY OF THE INVENTION

The present invention provides an improved open-headed bone screw including a reverse angled threadform with anti-splay clearance between threads on a closure member and threads within arms forming the open head and further including extended length arms with weakened areas to enable extensions of the arms to be broken off. The threadform has variations in embodiments that include parallel load flank pairs on the male and female threads and non-parallel load flank pairs. With the parallel load flanks, the thread stresses are applied substantially equally to the male and female threads. For parallel load flanks and a given equal cross sectional area of the male and female threads, the female threads tend to be stronger than the male threads.

Additionally, the present invention provides configurations of threadforms or thread structures which control the relative loading or proportioning of stresses between the threads on threaded members and threaded bores, such as within an open bone screw head and on a corresponding closure plug. Such control of loading can be done to selectively balance or equalize the joint stresses applied to the head and closure structures or to control which of the guide and advancement structures is more likely to fail first.

In general, for threads of a given cross sectional area and similar shape and with parallel load flanks, the receptacle or female thread is somewhat stronger than the closure or male thread. Each circumferential increment of the thread resembles a short cantilever beam, supported at one end and free or unsupported at the opposite end. For a given pair of engaged thread increments, the supported region of the receptacle thread has a greater circumference than the free region thereof while, in contrast, the supported region of the closure thread has less circumference than the free region. Thus, for a given circumferential length of thread, the receptacle thread has a longer connection region than the closure thread.

Under some circumstances, it is desirable to effectively equalize the relative strengths of the receptacle thread and the closure thread, for example to lower the likelihood of failure of either thread. Under other circumstances, it might be desirable to control which thread is likely to fail first. In general for helically joined elements in which one element is implanted in tissue such as bone, it is preferable for the thread of the non-implanted element to fail rather than the thread of the implanted element, to avoid removal and replacement of the implanted element. In the case of an implanted, open-headed bone screw receiving a closure plug, it is preferable that the thread of the closure fail before the thread of the receptacle. In the case of a bone screw having an externally threaded head over which an internally threaded nut or cap is placed, it is preferable that the internal or female thread of the nut or cap fail before the external or male thread of the head.

On threads with load flanks which converge outwardly from the helical axes, peak or crest regions of the inner threads of the closure member engage root regions of the bone screw head. Such an arrangement increases an effective moment arm of engagement of the closure thread and decreases an effective moment arm of the thread of the screw head, relative to a threadform configuration having parallel load flanks. Such a configuration with outwardly converging load flanks applies a greater proportion of the joint stress on the connection region of the closure thread than of the thread of the screw head when the closure is strongly torqued within the screw head so that if one of the thread fails, it is more likely to be the closure thread than the thread of the screw head.

Conversely, on threads with load flanks which diverge outwardly from the helical axes, peak or crest regions of the outer threads of the screw head engage root regions of the inner thread of the closure member. In this arrangement, the effective moment arm of engagement of the outer threads is increased while that of the inner thread of the closure member is decreased. Such an arrangement can be used to effectively equalize the joint stress between the closure thread and the head thread or to place a greater proportion of the joint stress on the screw head thread, depending on the angular difference between the load flanks.

Because of the reverse angled configuration of the load flanks of the threadforms of the present invention, the arms of the bone screw tend to be drawn inwardly toward the helical axis of the head and closure threads, particularly when there is resistance to threading the closure member into the head of the bone screw. When the closure member engages the rod within the channel and is torqued against resistance by the rod, it is possible for the arms to be drawn in to the point that the threads are deformed by mutual interference. Ultimately, when the closure member is torqued to clamp the rod at the seat of the channel, it is possible for the threads to interfere to the point of seizing or galling of the surfaces of the threads. In such a circumstance, any unthreading of the closure member may be very difficult.

To reduce the possibility of such thread deformation and seizing, the present invention provides anti-splay clearance between portions of the threads to enable the threads to flex somewhat without being permanently deformed. It is desirable for the closure member to be torqued to the point that the load flanks of the threads are in a situation of high static friction to thereby reliably clamp the rod without seizing. Such static friction can be overcome should it become necessary to unthread the closure member. In contrast, if the threads of the closure member and the arms become seized, it will be very difficult to remove the closure member without damaging the implanted screw head.

With threadforms having angular peak regions but not crest surfaces, the anti-splay clearance can be provided between the stab flanks. Such anti-splay clearance between the stab flanks is in addition to the small amount of clearance that is normally provided between the stab flanks of the closure and head threads. With threads having outer cylindrical crest surfaces or other crest surface shapes, the anti-splay clearance is provided between the crest surfaces and the corresponding root surfaces, with additional anti-splay clearance between the stab flanks of the threads. The anti-splay clearance is desirable regardless of the relative angular relationships of the load flanks of these threads.

In order to facilitate capturing a spinal fixation rod which is initially spaced a considerably distance from the seat of a channel of a bone screw which is intended to receive the rod, the arms of the open-headed bone screw are provided with break-off extensions. The increased length of the arms enables the rod to be captured within the channel with less resistance of the rod than would be possible closer to the rod seat within the bone screw channel. The threaded closure is then threaded into the channel between the arms and used to urge the rod toward the seat. Once the rod is fully seated and clamped into place, the arm extensions can be separated from the more proximate portions of the arms by breaking them at weakened areas or notches formed at break points along the arms. The anti-splay features of the reverse angled threads of the present invention are particularly useful in combination with the increased lengths of the arms since such elongated arms tend to be more flexible than the proximate portions of the arms. With conventional V-threads, the increased flexibility of the arm extensions in combination with the outward camming action of the V-threads increases the difficulty in “reducing” or urging the rod toward the channel seat because of tendencies of the closure threads to slip out of engagement with the threads of the arms due to splaying of the arms. What is needed is a threadform which reduces, counteracts, or avoids tendencies of conventional V-threads to cause splaying of the arms of an open-headed bone screw during engagement of the closure with the arms.

### OBJECTS AND ADVANTAGES OF THE INVENTION

Therefore, objects of the present invention include: providing an improved threadform; providing such an improved threadform which has particularly advantageous application on an open headed lightweight and low profile medical implant; providing a threadform for such an implant which has a pair of spaced arms and the closure closes between the arms to clamp structure such as a spinal fixation rod therein; providing such a threadform which is a reverse angled threadform that resists tendencies of the arms to splay or separate during insertion of the closure, to thereby reduce the likelihood of failure of the implant and closure system during use; providing such a threadform which enables the closure to be installed at comparatively high torques to thereby secure the closure in the receiver channel and in certain embodiments to also lock a rod member in the open head of the implant where the closure engages and is urged against the rod by rotation in a receiver channel of the remainder of the implant; providing such a thread or threadform including clearance between elements of the threads to avoid galling and/or distortion of the threads when a closure is applied at high levels of torque within the head of the implant; providing a configuration of such a threadform with angular peaks in which the anti-splay clearance is implemented as space between stab flanks of the threads; providing a configuration of such a threadform with cylindrical crest and root surfaces in which the anti-splay clearance is implemented as space between the crest and root surfaces of the threads; providing such a threadform in which the threads of inner and outer members are proportioned and configured in such a manner as to control the relative levels of stress which are applied to the inner and outer threads when the threaded joint is strongly torqued; providing such a threadform in which the load flanks are substantially parallel; providing such a threadform in which the load flanks diverge in a radially outward direction; providing such a threadform in which the load flanks converge in a radially outward direction; providing such a threadform which can be formed relatively economically using appropriate metal forming technologies; and providing reverse angled threadforms with anti-splay clearance, particularly for implant and bone fixation hardware, which are economical to manufacture, which are secure and efficient in use, and which are particularly well adapted for their intended usage.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an enlarged side elevational view of a rod capturing bone screw incorporating a reverse angled threadform with anti-splay clearance which embodies the present invention, with portions of arms of the screw head broken away to illustrate details of the threadform.

FIG. 2 is a view similar to FIG. 1 and illustrates the bone screw with a closure member in clamped engagement with a spinal fixation rod and with arm extensions and an installation head broken off.

FIG. 3 is a greatly enlarged sectional view of a reverse angled threadform of the present invention including angular peak regions and in which the load flanks are parallel.

FIG. 4 is a view similar to FIG. 3 and shows the reverse angled threadform with parallel load flanks in a situation of high torque.

FIG. 5 is a greatly enlarged sectional view of a reverse angled threadform of the present invention including angular peak regions and in which the load flanks diverge outwardly.

FIG. 6 is a view similar to FIG. 5 and shows the outwardly diverging load flanks of the reverse angled threadform in a situation of high torque.

FIG. 7 is a greatly enlarged sectional view of a reverse angled threadform of the present invention including angular peak regions and in which the load flanks converge outwardly.

FIG. 8 is a view similar to FIG. 7 and shows the outwardly converging load flanks of the reverse angled threadform in a situation of high torque.

FIG. 9 is a greatly enlarged sectional view of a reverse angled threadform of the present invention including cylindrical crest and root surfaces and in which the load flanks are parallel.

FIG. 10 is a view similar to FIG. 9 and shows the reverse angled threadform with parallel load flanks in a situation of high torque.

FIG. 11 is a greatly enlarged sectional view of a reverse angled threadform of the present invention including cylindrical crest and root surfaces and in which the load flanks diverge outwardly.

FIG. 12 is a view similar to FIG. 11 and shows the reverse angled threadform with outwardly diverging load flanks in a situation of high torque.

FIG. 13 is a greatly enlarged sectional view of a reverse angled threadform of the present invention including cylindrical crest and root surfaces and in which the load flanks converge outwardly.

FIG. 14 is a view similar to FIG. 13 and shows the reverse angled threadform with outwardly converging load flanks in a situation of high torque.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 1 generally designates a reverse angled threadform with anti-splay clearance which embodies the present invention. The threadform 1 is incorporated in a spinal fixation anchor 2 formed by an open headed bone screw 3 and a closure 4 that is received in the bone screw 3 to clamp and thereby anchor a spinal fixation rod 5. Although the threadform 1 is foreseen to have wider and more diverse applications than medical implants, the variations in configurations of the threadform 1 of the present invention will be described herein in connection with the medical implant 6 formed by the bone screw 3 and closure 4. It is also foreseen that the bone screw can be cannulated and have a polyaxial head, as will be described in more detail below.

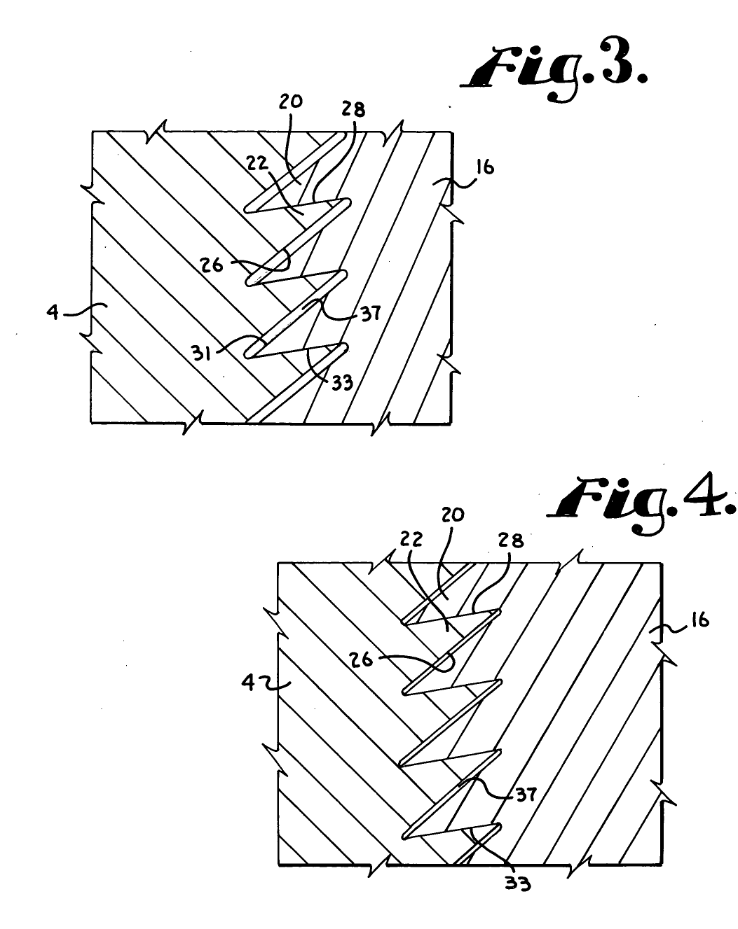

The illustrated bone screw 3 includes a threaded shank 14 and a pair of spaced apart arms 16 which are joined to the shank 14 to form a seat 18 to receive the rod 5. The illustrated arms 16 may include break-off extensions 17 formed by weakened regions 19 to enable capture of a rod 5 at a greater height from the rod seat 18. The extensions 17 can be separated after the rod 5 is reduced by advancement of the closure 4 to result in the low profile implant 6 shown in FIG. 2. The threaded shank 14 is adapted for threaded implanting into a bone 15, such as a vertebra. Reverse angled threads 20 are formed or cut into inner surfaces of the arms 16. The threads 20 are referred to herein as outer threads since they are formed on the relative outer members 16. The cylindrical closure 4 is sized diametrically to be received between the arms 16 and has threads 22 formed or cut into an outer surface thereof. The closure 4 may include a torque limiting break-off head 12 which separates from the closure 4 at a selected level of torque between the closure 4 and the arms 16. The threads 22 are referred to as inner threads since they are formed on the relatively inner member 4. The threads 20 and 22 are compatible and engage the closure 4 to be threaded into engagement with the rod 5 to thereby clamp the rod 5 between the closure 4 and the rod seat 18.

In particular, the threads 20 and 22 are reverse angled threadforms with anti-splay clearance incorporated therebetween to accommodate inward drawing of parts of the outer member, such as arms 16, in response to high levels of torque while minimizing permanent deformation of the threads 20 and 22 or galling of the threads.

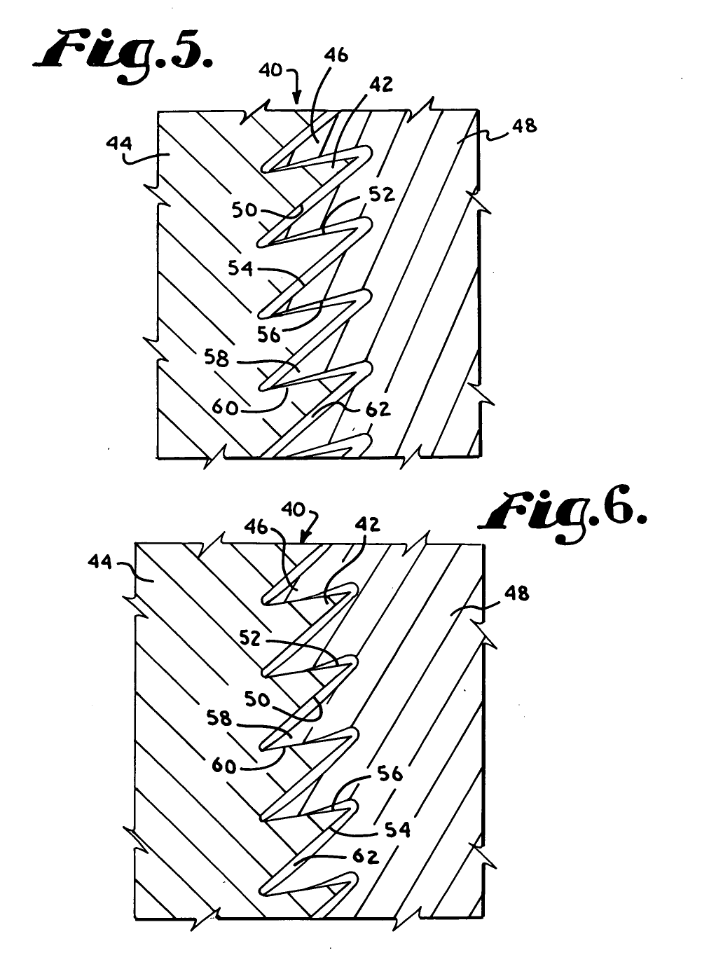

Referring to FIGS. 3 and 4, the enlarged illustrations show the inner threads 22 of the closure member 4 engaged with the outer threads 20 of an arm 16 of the screw 3. The inner threads 22 have leading stab flanks 26 and trailing load flanks 28. The leading and trailing nature of the flanks 26 and 28 is referenced to a direction of travel of the closure 4 (indicated by arrow 29 in FIGS. 3 and 4) between the arms as the closure 4 is rotated in a rod engaging or clockwise direction. Similarly, the outer threads 20 have leading stab flanks 31 and trailing load flanks 33. When the closure is advanced into a position between the arms 16, the inner and outer load flanks 28 and 33 engage.

The threads 20 and 22 are referred to as reverse angled threads because the surfaces of the inner load flanks 28 form acute angles with the axis of rotation 34 (FIG. 2) of the closure 4, while the surfaces of the outer load flanks 33 form complementary obtuse angles with the axis 34. The angular relationships of the load flanks 28 and 33 to the axis 34 is opposite that of conventional “forward” angled V-threads. With conventional V-threads, when advancing movement of the closure 4 is prevented by contact with the rod 5, the reaction of the arms 16 to continued torque on the closure 4 would be to be spread or splayed by cooperative camming action of such V-threads. However, with the illustrated reverse angled threads 20 and 22, the reaction of the arms 16 to such continued torque with linear advancement of the closure 4 blocked is for the arms 16 to be drawn inward toward the axis 34, that is, in an anti-splay direction. The advantage of reverse angled threads, particularly in an application such as the open headed bone screw 3 and closure 4 is that high levels of torque do not have a tendency to cause the threads 22 of the closure 4 to slip past the threads 20 of the arms 16, as could happen with conventional V-shaped threads.

Typically, there is at least a small amount of clearance between the stab flanks of engaged threads to facilitate relative movement between the load flanks. However, with reverse angled threads, such as the threads 20 and 22, inward movement of the arms 16 can cause engagement of the stab flanks 26 and 31 in addition to the engagement of the load flanks 28 and 33. High levels of torque between the closure 4 and the arms 16 can result in strong inward movement of the arms 16, thereby causing possible permanent deformation of portions of the threads 20 and 22 and possibly galling between the threads, complicating subsequent removal of the closure 4 should such removal be required.

In the present invention, an anti-splay clearance 37 is provided between the reverse angled threads 20 and 22 to prevent possible deformation and/or galling between the threads when the closure 4 is strongly torqued into contact with the rod 5. The anti-splay clearance 37 enables the closure 4 to be strongly torqued into contact with the rod 5 with engagement between the threads 20 and 22 restricted to engagement between the load flanks 28 and 33.

The reverse angled threads 20 and 22 illustrated in FIGS. 3 and 4 have thread peaks formed by simple angular intersection of stab flanks 26 and 31 respectively with load flanks 28 and 33. With this arrangement, the anti-splay clearance is implemented as an increased clearance between the stab flanks 26 and 31 of the threads 20 and 21. With other configurations of threads and similar structures, such as various types of guide and advancement flanges, anti-splay clearances may be formed between other components of such threads and structures, as will be described in more detail below.

On the threads 20 and 22 illustrated in FIGS. 3 and 4, the load flanks 28 and 33 are oriented in parallel relation such that axial stresses exerted on the threads 20 and 22 resulting from high levels of torque between the closure 4 and the arms 16 are distributed relatively evenly along the load flanks 28 and 33.

Incremental circumferential sectors of the threads 20 and 22 function somewhat like cantilever beams in that they are supported at a root end and are free at the crest end. For a given angular size of engaged increments and assuming the same profile area and depth of the threads 20 and 22, the outer increment of the outer thread 20 is slightly stronger than the inner increment of the inner thread 22. This is probably because the circumference of the root of the outer thread 20 is slightly longer than the circumference of the root of the inner thread 22. As a result, if one of the threads 20 or 22 is likely to fail in a high torque situation, with parallel load flanks 28 and 33, the inner thread 22 is more likely to be the one that fails. Where threaded attachments are to be made to implanted structure, if there is a possibility of failure of the threads under high torque conditions, it is preferable for the threads of the non-implanted element to fail rather than the threads of the implanted element to avoid the necessity of removal and replacement of the implanted element.

In the illustrated configuration of the implant 6 with the implanted bone screw 3 and internal closure 4, the inherent tendency of the outer threads 20 of the arms 16 to be stronger than the inner threads 22 of the closure 4 is beneficial. However, there are known configurations of open headed bone screws with threaded external closures in which the relatively weaker inner threads would be located on the implanted bone screw. Thus, there is a need for the capability of controlling the proportioning of axial stresses on the cooperating threads, depending on the circumstances of application of the threads. One possibility is to make the profile area of the preferred thread larger. The present invention provides an alternative solution.

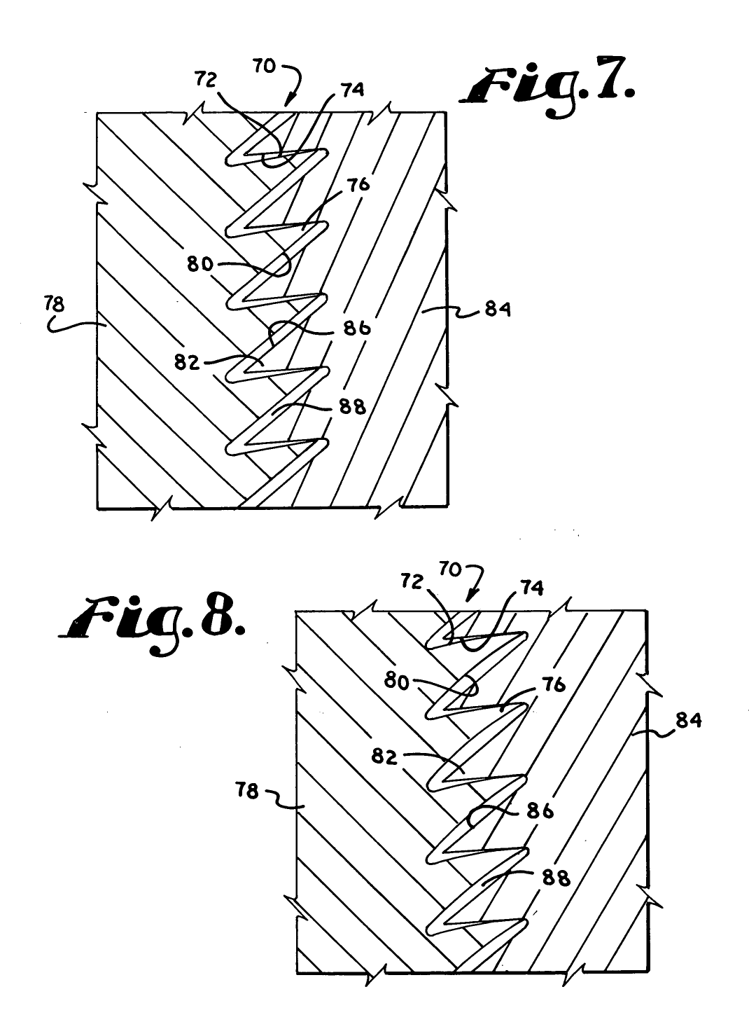

FIGS. 5 and 6 illustrate a reverse angled threadform 40 including inner threads 42 of an inner member 44, such as the closure member 4, and outer threads 46 of an outer member 48, such as an arm 16 of the bone screw 3. The inner threads 42 include leading stab flanks 50 and trailing load flanks 52. Similarly, the outer threads 46 include leading stab flanks 54 and trailing load flanks 56. The inner and outer load flanks 52 and 56 engage when the inner member 44 is advanced into the outer member 48. In particular, the inner and outer load flanks 52 and 56 diverge in an angular manner in a direction outward from the inner member 44 toward the outer member 48.

By this configuration, engagement between the threads 42 and 46 begins between a peak region 58 of the outer threads 46 and a root region 60 of the inner threads 42. The effect of this configuration of the threadform 40 is to concentrate axial stresses between the threads 42 and 46 at high torque at the strongest part of the inner threads 42, the root region 60, and to end load the stress to the outer thread 46 through the moment arm of the depth of the outer thread 46. Such an arrangement tends to make the inner threads 42 relatively stronger than the outer threads 46, which is beneficial in some thread applications. The threadform 40 is provided with an anti-splay clearance 62 between the stab flanks 50 and 54 which provides the same benefits to the threadform 40 as the clearance 37 of the threadform 1.

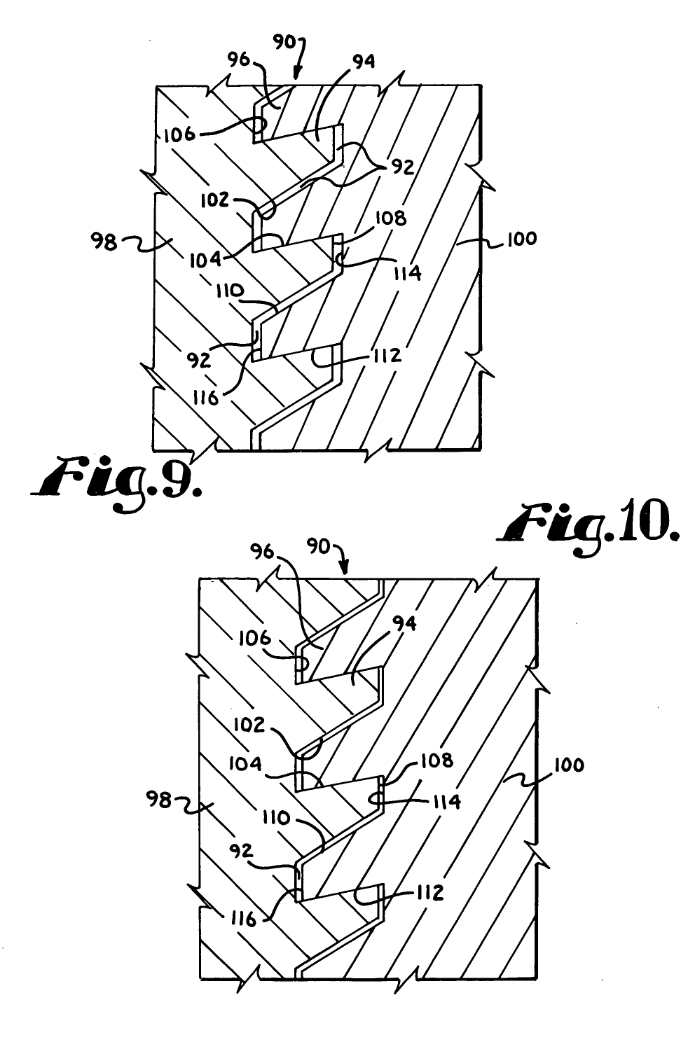

FIGS. 7 and 8 illustrate a reverse angled threadform 70 with outwardly converging load flanks 72 and 74, in low torque (FIG. 7) and high torque (FIG. 8) conditions. The threadform 70 includes inner threads 76 of an inner member 78 with the trailing load flanks 72 and leading stab flanks 80. Similarly, the threadform 70 includes outer threads 82 of an outer member 84 having the trailing load flanks 74 and leading stab flanks 86. The load flanks 72 and 74 converge in an outer direction from the inner member 78 toward the outer member 84.

The effect of outward convergence of the load flanks 72 and 74 is to initiate engagement between the threads 76 and 82 at the root regions of the outer threads 82 and the peak regions of the inner threads 76. By this arrangement, axial stress between the inner and outer members 78 and 84 is applied at the root regions or strongest parts of the outer threads 82 and through the moment arms of the depths of the inner threads 76. Thus, proportioning of axial stress on the threadform 70 is controlled by effectively applying a greater proportion of such stress on the inner threads 76, with less stress on the outer threads 82, such that if the threadform 70 should fail from high levels of torque, it is more likely that the inner threads 76 would fail.

The threadform 70 is provided with anti-splay clearance 88 between the stab flanks 80 and 96 to enable portions of the outer member 84 to be drawn inwardly in reaction to high levels of torque between the inner and outer members 78 and 84 without permanent deformation of the threads 76 and 82. As illustrated in FIG. 8, high levels of torque between the inner and outer members 78 and 84 can cause some temporary deformation of the threads 76. The degree and permanence of such deformation is determined by various factors, including the relative levels of torque between the inner and outer members 78 and 84 and the materials from which the members 78 and 84 are constructed.

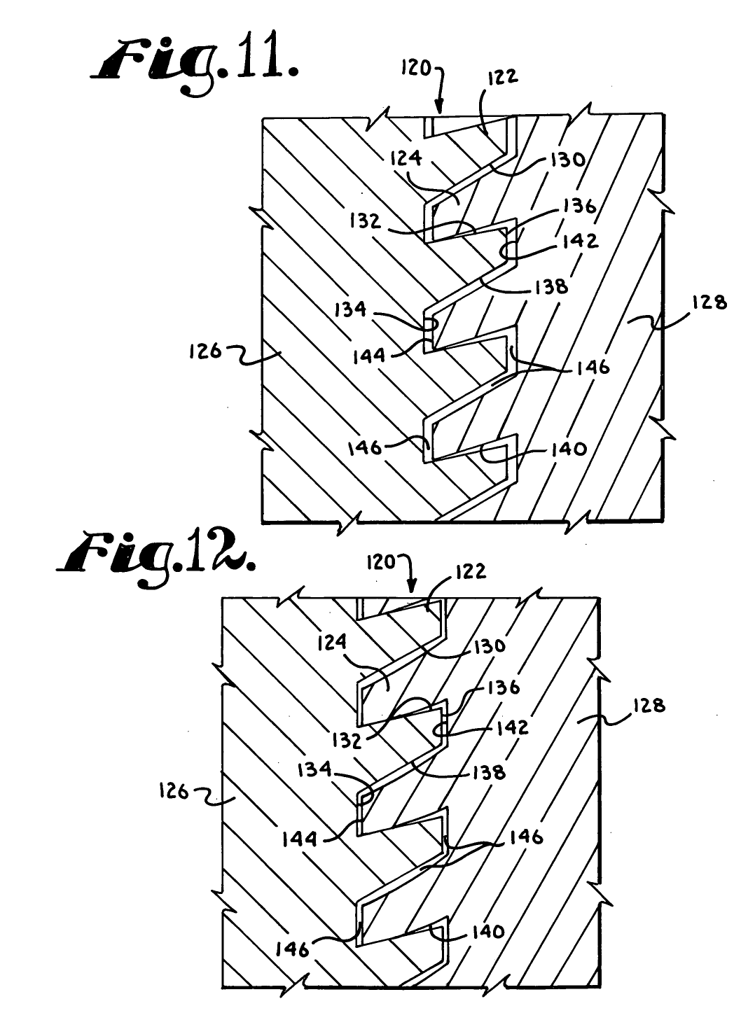

FIGS. 9 and 10 illustrate a modified reverse angled threadform 90 of the present invention, including anti-splay clearance 92. The threadform 90 includes inner and outer threads 94 and 96 respectively of inner and outer members 98 and 100. The inner thread 94 includes leading inner stab flanks 102, trailing inner load flanks 104, cylindrical inner root surfaces 106, and cylindrical inner crest surfaces 108. Similarly, the outer thread 96 includes leading outer stab flanks 110, trailing outer load flanks 112, cylindrical outer root surfaces 114, and cylindrical outer crest surfaces 116.

In the threadform 90, the anti-splay clearance 92 is formed between the inner and outer stab flanks 102, between the inner root surfaces 106 and outer crest surfaces 116, and between the inner crest surfaces 108 and outer root surfaces 114. The anti-splay clearance 92 allows portions of the outer member 100 to be drawn inwardly somewhat in reaction to high levels of torque between the inner and outer members 98 and 100 without permanent deformation of the threads 94 and 96. In the illustrated threadform 90, the load flanks 104 and 112 are substantially parallel, whereby axial stress between the inner and outer members 98 and 100 is proportioned substantially equally between the inner and outer threads 94 and 96.

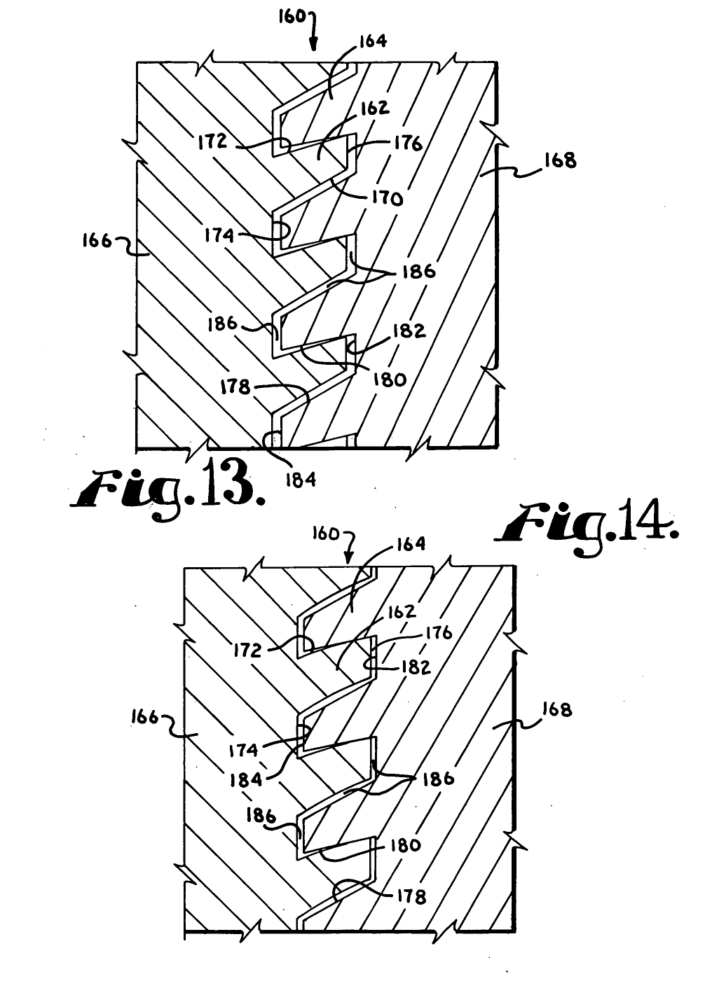

FIGS. 11 and 12 illustrate an additional modified embodiment of a reverse angled threadform 120 according to the present invention. The threadform 120 includes inner and outer threads 122 and 124 respectively of inner and outer members 126 and 128. The inner thread 122 includes leading inner stab flanks 130, trailing inner load flanks 132, cylindrical inner root surfaces 134, and cylindrical inner crest surfaces 136. Similarly, the outer thread 124 includes leading outer stab flanks 138, trailing outer load flanks 140, cylindrical outer root surfaces 142, and cylindrical outer crest surfaces 144. An anti-splay clearance 146 is formed between the inner and outer stab flanks 130 and 138, between the inner root surfaces 134 and the outer crest surfaces 144, and between the inner crest surfaces 136 and the outer root surfaces 142. As illustrated in FIGS. 11 and 12, the inner and outer load flanks 132 and 140 diverge outwardly in a radial direction from the inner member 126 toward the outer member 128 to thereby apply axial stress between the inner and outer members 126 and 128 at the root region of the inner thread 122 and through the moment arm of the depth of the outer thread 124, thereby increasing the relative strength of the inner thread 122 and decreasing the relative strength of the outer thread 124.

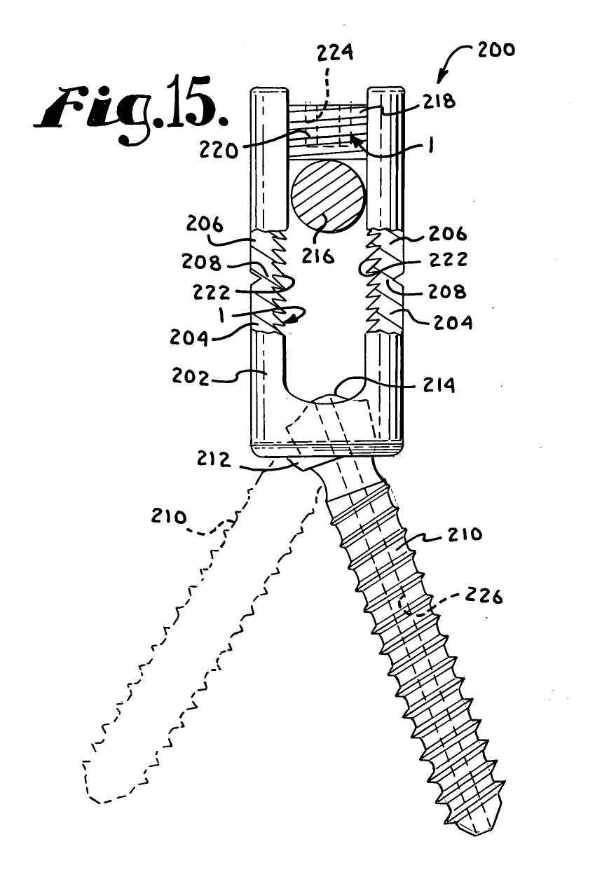

FIGS. 13 and 14 illustrate a further embodiment of a threadform 160 according to the present invention. The threadform 160 includes inner and outer threads 162 and 164 respectively of inner and outer members 166 and 168. The inner thread 162 includes leading inner stab flanks 170, trailing inner load flanks 172, cylindrical inner root surfaces 174, and cylindrical inner crest surfaces 176. Similarly, the outer thread 164 includes leading outer stab flanks 178, trailing outer load flanks 180, cylindrical outer root surfaces 182, and cylindrical outer crest surfaces 184. In the threadform 160, an anti-splay clearance 186 is formed between the inner and outer stab flanks 170 and 178, between the inner root surfaces 174 and outer crest surfaces 184, and between the inner crest surfaces 176 and outer root surfaces 182. In the threadform 160, the inner and outer load flanks 172 and 180 converge outwardly in a radial direction from the inner member 166 toward the outer member 168 to thereby apply axial stresses resulting from high levels of torque between the inner and outer members 166 and 168 at the root region of the outer threads 164 through the moment arms of the inner threads 162, whereby the outer threads 164 are relatively strengthened and the inner threads are relatively weakened.

FIG. 15 illustrates the incorporation of the reverse angled threadform 1 with anti-splay clearance of the present invention into a polyaxial type of bone screw assembly 200. The assembly 200 generally includes a U-shaped receiver 202 formed by spaced apart arms 204 with break-off extensions 206 connected thereto by weakened areas 208 and a threaded shank 210 joined to the receiver 202 by polyaxial retaining and articulating structure generally represented by a retaining ring 212. The structure or ring 212 has a spherical outer surface which engages a similar surface within the receiver 202 to enable the shank 210 to be positioned at any desired angle relative to the receiver 202 within a selected range of angles.

The shank 210 has a capture end 214 at a proximal end thereof which is adapted for engagement by a rod or rod-like spinal fixation member 216 to thereby clamp the rod-like member 216 between the capture end 214 and a cylindrical closure 218 which also fixes and secures the angular relationship of the shank 210 relative to the receiver 202. The closure 218 has inner threads 220 while the inner surfaces of the arms, including the extensions 206, have outer threads 222 formed thereon. The threads 220 and 222 may be any of the reverse angled threadforms illustrated in FIGS. 2-14 and incorporate suitable anti-splay clearances therein.

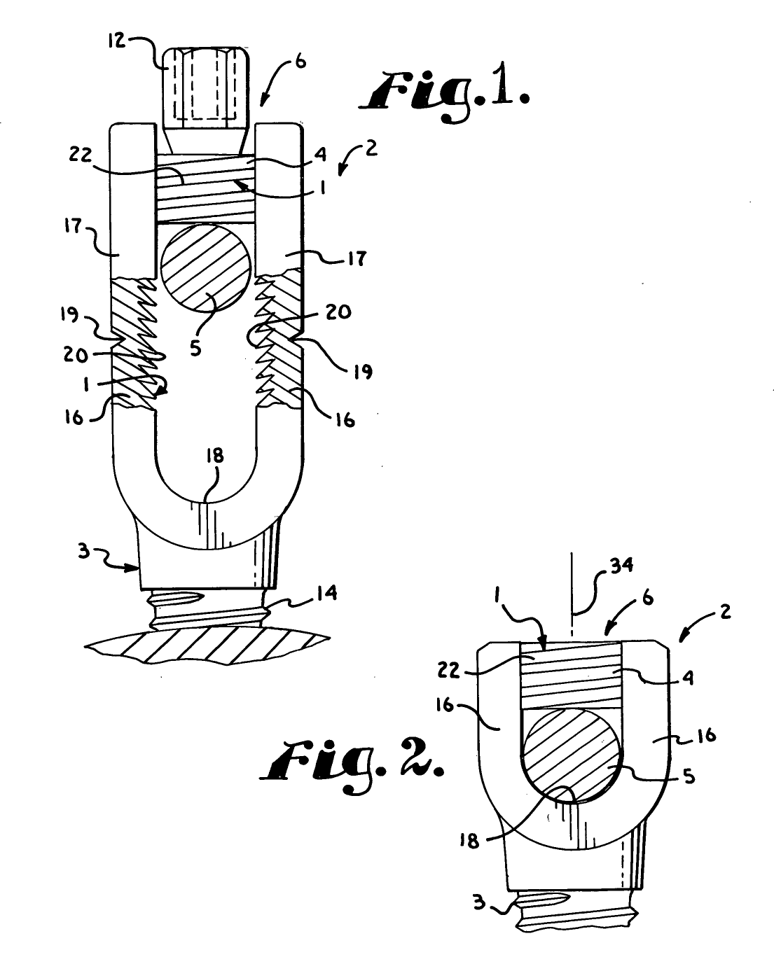

The illustrated closure 220 is provided with a non-round opening 224, such as an Allen or Torx type of opening, to receive a similarly shaped tool (not shown) to advance the closure 220 into the receiver 202. Alternatively, the closure 220 could be provided with a torque limiting break-off head similar to the head 12 shown in FIG. 1. The illustrated shank 210 is a cannulated shank, having a cannula or cannulation 226 bored therethrough, to receive a guide wire or elongated guide member therethrough to thereby facilitate use of the assembly 200 in percutaneous spinal fixation procedures. Alternatively, the shank 210 can be formed as a non-cannulated shank. Further details of polyaxial bone screws with cannulated threaded shanks can be obtained by reference to U.S. Pat. No. 6,716,214.

It is to be understood that while certain forms of the present invention have been illustrated and described herein, it is not to be limited to the specific forms or arrangement of parts described and shown. In particular, it is foreseen that the reverse angled threadform 1 with anti-splay clearance can be advantageously employed with various hooks, connectors, both cannulated and non-cannulated polyaxial screws, and other types of spinal implants.

What is claimed and desired to be secured by Letters Patent is as follows:

1. In a medical device threadform for advancing an inner member into an opening within an outer member and including an inner thread on said inner member and an outer thread within said opening, said outer member having a tendency to splay in response to torquing said inner member within said opening, the improvement comprising:
(a) said inner thread and said outer thread being configured to draw said outer member toward said inner member in response to torquing said inner member within said opening;
(b) said inner thread having an inner leading flank and an inner trailing flank, said outer thread having an outer leading flank and an outer trailing flank, said inner and outer leading flanks being substantially disengaged when said inner member is advanced into said outer member;
(c) said inner and outer trailing flanks are mutually engaging when said inner member is advanced into said outer member;
(d) said inner and outer trailing flanks have respective facing surfaces that are substantially non parallel when said inner member is advanced into said outer member;
(e) said inner thread and said outer thread being configured to provide an anti-splay clearance therebetween upon mutual engagement thereof to thereby facilitate said outer member being drawn toward said inner member in response to said torquing said inner member within said opening; and
(f) said anti-splay clearance is formed, at least partially, between said inner and outer leading flanks when said inner member is advanced into said outer member.

2. The threadform according to claim 1 wherein said trailing flank surfaces mutually diverge in an outward direction from said inner member.

3. A threadform as set forth in claim 1 wherein:
(a) said inner and outer trailing flanks are respectively inner and an outer load flanks, said inner and outer load flanks mutually engaging when said inner member is advanced into said outer member; and
(b) said inner and outer load flanks have respective elements which mutually diverge in an outward direction from said inner member to said outer member when said inner member is advanced into said outer member.

4. A threadform as set forth in claim 1 wherein:
(a) said inner and outer trailing flanks are respectively inner and outer load flanks, said inner and outer load flanks mutually engaging when said inner member is advanced into said outer member; and
(b) said inner and outer load flanks have respective elements which mutually converge in an outward direction from said inner member to said outer member when said inner member is advanced into said outer member.

5. A threadform as set forth in claim 1 wherein:
(a) said inner thread has an inner root surface and an inner crest surface;
(b) said outer thread has an outer root surface and an outer crest surface; and
(c) said anti-splay clearance is formed partially between said inner root surface and said outer crest surface and between said inner crest surface and said outer root surface.

6. A threadform as set forth in claim 1 wherein:
(a) said outer thread is formed on inner surfaces of arms of a U-shaped bone screw head which is adapted for threaded implanting in a bone, said U-shaped head defining a channel adapted to receive a spinal fixation member; and
(b) said inner thread is formed on an outer surface of a cylindrical closure threadedly receivable within said U-shaped head to thereby clamp said spinal fixation member within said head.

7. A threadform as set forth in claim 1 wherein:
(a) said outer thread is formed on inner surfaces of arms of a U-shaped bone screw head which is adapted for threaded implanting in a bone, said U-shaped head defining a channel adapted to receive a spinal fixation member;
(b) said arms of said head include elongated break-off extensions connected respectively to said arms by weakened regions and including said outer thread formed on inner extension surfaces of said extensions;
(c) said inner thread is formed on an outer surface of a cylindrical closure threadedly receivable within said U-shaped head to thereby clamp said spinal fixation member within said head; and
(d) said extensions are separated from said arms after said spinal fixation member is clamped in said channel to thereby reduce a profile of said bone screw head.

8. A threadform as set forth in claim 1 wherein:
(a) said outer thread is formed on inner surfaces of arms of a U-shaped bone screw receiver of a polyaxial bone screw which is adapted for articulated connection to a bone, said U-shaped head defining a channel adapted to receive a spinal fixation member; and including
(b) the screw has a threaded shank connected to said receiver by articulating structure to provide said articulated connection of said receiver to said bone; and
(c) said inner thread is formed on an outer surface of a cylindrical closure threadedly receivable within said U-shaped receiver to thereby clamp said spinal fixation member within said receiver.

9. A threadform as set forth in claim 1 wherein:
(a) said outer thread is formed on inner surfaces of arms of a U-shaped bone screw receiver of a polyaxial bone screw which is adapted for articulated connection to a bone, said U-shaped head defining a channel adapted to receive a spinal fixation member;
(b) a threaded shank of a polyaxial bone screw is connected to said receiver by articulating structure to provide said articulated connection of said receiver to said bone, said shank being cannulated for reception of an elongated guide member therethrough; and
(c) said inner thread is formed on an outer surface of a cylindrical closure threadedly receivable within said U-shaped receiver to thereby clamp said spinal fixation member within said receiver.

10. A medical device threadform for guiding and advancing an inner member into an opening within an outer member in a selected direction of advancement of said inner member into said outer member in response to rotation of said inner member into said opening in a selected direction of rotation and comprising:
(a) an inner thread extending helically about said inner member relative to an inner helical axis extending through said inner member;
(b) an outer thread extending helically about said opening within said outer member relative to an outer helical axis extending through said opening;
(c) said inner thread and said outer thread being configured and cooperating in such a manner as to tend to draw said outer member toward said outer axis upon torquing said inner member within said outer member; and
(d) said inner thread and said outer thread being shaped and dimensioned in such a manner as to form an anti-splay clearance therebetween upon mutual engagement thereof to thereby facilitate said outer member being drawn toward said outer axis in response to said torquing said inner member within said outer member; wherein
(e) each of said inner and outer threads has a leading flank and a trailing flank, said inner and outer trailing flanks being substantially non parallel in an outward direction, and said inner and outer leading flanks being substantially disengaged when said inner member is advanced into said outer member; and
(f) said anti-splay clearance is formed, at least partially, between said inner and outer leading flanks as said inner member is advanced into said outer member.

11. A threadform as set forth in claim 10 wherein:
(a) said inner thread includes alternating inner crests and inner roots;
(b) said outer thread includes alternating outer crests and outer roots; and
(c) said inner thread and outer thread are configured in such a manner that said anti-splay clearance separates said inner crests from said outer roots and said inner roots from said outer crests.

12. A threadform as set forth in claim 10 wherein:
(a) said inner thread has an inner trailing flank which forms an acute angle with said inner axis and faces generally toward said inner axis; and
(b) said outer thread has an outer trailing flank which forms an obtuse angle with said outer axis and faces generally away from said outer axis.

13. A threadform as set forth in claim 10 wherein:
(a) said inner thread and said outer thread are shaped and dimensioned in such a manner that upon advancement of said inner member into said outer member, said inner load flank and said outer load flank diverge relative to one another in an outward radial direction relative to said inner axis.

14. A threadform as set forth in claim 10 wherein:
(a) said inner thread and said outer thread are shaped and dimensioned in such a manner that upon advancement of said inner member into said outer member, said inner load flank and said outer load flank converge relative to one another in an outward radial direction relative to said inner axis.

15. A threadform as set forth in claim 10 wherein:
(a) said inner thread has an inner root region and an inner peak region positioned radially outward of said inner root region relative to said inner axis;
(b) said outer thread has an outer root region and an outer peak region positioned radially inward of said outer root region relative to said outer axis; and
(c) said inner thread and said outer thread have such respective shapes and dimensions that, upon advancement of said inner member into said outer member, said outer peak region engages said inner root region prior to said inner peak region engaging said outer root region.

16. A threadform as set forth in claim 10 wherein:
(a) said inner thread has an inner root region and an inner peak region positioned radially outward of said inner root region relative to said inner axis;
(b) said outer thread has an outer root region and an outer peak region positioned radially inward of said outer root region relative to said outer axis; and
(c) said inner thread and said outer thread have such respective shapes and dimensions that, upon advancement of said inner member into said outer member, said inner peak region engages said outer root region prior to said outer peak region engaging said inner root region.

17. A threadform as set forth in claim 10 wherein:
(a) said inner thread has an inner root region positioned at an inner root radius relative to said inner axis and an inner peak region positioned radially outward of said inner root region at an inner peak radius relative to said inner axis;
(b) said outer thread has an outer root region positioned at an outer root radius relative to said outer axis and an outer peak region positioned radially inward of said outer root region at an outer peak radius relative to said outer axis; and
(c) said outer peak radius exceeds said inner root radius and said outer root radius exceeds said inner peak radius to thereby form said anti-splay clearance between said inner thread and said outer thread.

18. A threadform as set forth in claim 10 wherein:
(a) said inner thread has an inner root region positioned at an inner root radius relative to said inner axis and an inner peak region formed by a convergence between an inner trailing flank and an inner leading flank, said inner peak region being positioned radially outward of said inner root region at an inner peak radius relative to said inner axis;
(b) said outer thread has an outer root region positioned at an outer root radius relative to said outer axis and an outer peak region formed by convergence of an outer trailing flank and an outer leading flank, said outer peak region being positioned radially inward of said outer root region at an outer peak radius relative to said outer axis; and
(c) said outer peak radius exceeds said inner root radius and said outer root radius exceeds said inner peak radius to thereby form said anti-splay clearance between said inner leading flank and said outer leading flank.

19. A threadform as set forth in claim 10 wherein:
(a) said outer thread is formed on inner surfaces of arms of a U-shaped bone screw head which is adapted for threaded implanting in a bone, said U-shaped head defining a channel adapted to receive a spinal fixation member; and
(b) said inner thread is formed on an outer surface of a cylindrical closure threadedly receivable within said U-shaped head to thereby clamp said spinal fixation member within said head.

20. A threadform as set forth in claim 10 wherein:
(a) said outer thread is formed on inner surfaces of arms of a U-shaped bone screw head which is adapted for threaded implanting in a bone, said U-shaped head defining a channel adapted to receive a spinal fixation member;
(b) said arms of said head include elongated break-off extensions connected respectively to said arms by weakened regions and including said outer thread formed on inner extension surfaces of said extensions;
(c) said inner thread is formed on an outer surface of a cylindrical closure threadedly receivable within said U-shaped head to thereby clamp said spinal fixation member within said head; and
(d) said extensions are separated from said arms after said spinal fixation member is clamped in said channel to thereby reduce a profile of said bone screw head.

21. A threadform as set forth in claim 10 wherein:
(a) said outer thread is formed on inner surfaces of arms of a U-shaped bone screw receiver of a polyaxial bone screw which is adapted for articulated connection to a bone, said U-shaped head defining a channel adapted to receive a spinal fixation member;
(b) a threaded shank of a polyaxial bone screw is connected to said receiver by articulating structure to provide for said articulated connection of said receiver to said bone; and
(c) said inner thread is formed on an outer surface of a cylindrical closure threadedly receivable within said U-shaped receiver to thereby clamp said spinal fixation member within said receiver.

22. A threadform as set forth in claim 10 wherein:
(a) said outer thread is formed on inner surfaces of arms of a U-shaped bone screw receiver of a polyaxial bone screw which is adapted for articulated connection to a bone, said U-shaped head defining a channel adapted to receive a spinal fixation member;
(b) a threaded shank of a polyaxial bone screw is connected to said receiver by articulating structure to provide for said articulated connection of said receiver to said bone, said shank being cannulated for reception of an elongated guide member therethrough; and
(c) said inner thread is formed on an outer surface of a cylindrical closure threadedly receivable within said U-shaped receiver to thereby clamp said spinal fixation member within said receiver.

23. A medical device threadform for guiding and advancing an inner member into an opening within an outer member in a selected direction of advancement of said inner member into said outer member in response to rotation of said inner member into said opening in a selected direction of rotation and comprising:
(a) an inner thread extending helically about said inner member relative to an inner helical axis extending through said inner member, said inner thread being formed by an inner trailing flank on a trailing side of said inner thread relative to said direction of advancement and an inner leading flank on a leading side of said inner thread relative to said direction of advancement;
(b) an outer thread extending helically about said opening within said outer member relative to an outer helical axis extending through said opening, said outer thread being formed by an outer trailing flank on a forward side of said outer thread relative to said direction of advancement and an outer leading flank on a reverse side of said outer thread relative to said direction of advancement;
(c) said inner trailing flank and said outer trailing flank being oriented in such respective directions and cooperatively engaging in such a manner as to tend to draw said outer member toward said outer axis upon torquing said inner member within said outer member; and
(d) said inner thread and said outer thread being shaped and dimensioned in such a manner as to form an anti-splay clearance at least partially between said inner and outer leading flanks upon mutual engagement thereof to thereby facilitate said outer member being drawn toward said outer axis in response to said torquing said inner member within said outer member; wherein
(e) said inner and outer leading flanks are substantially disengaged as said inner member is advanced into said outer member;
(f) said inner trailing flank forms an acute angle with said inner axis;
(g) said outer trailing flank forms an obtuse angle with said outer axis and faces generally away from said outer axis; and
(h) said inner and outer trailing flanks are substantially non parallel.

24. The threadform according to claim 23 wherein the inner and outer trailing surfaces diverge outwardly from the inner member when originally joined.

25. The threadform according to claim 23 wherein the inner and outer trailing surfaces converge outwardly from the inner member when originally joined.

26. In a medical device threadform for advancing an inner member into an opening within an outer member, the outer member having a tendency to splay in response to torquing the inner member within the opening comprising:
(a) an inner member thread including an inner leading flank, an inner trailing flank, an inner root surface and an outer crest surface; and
(b) an opening outer thread including an outer leading flank, an outer trailing flank, an outer root surface and an outer crest surface; wherein
(c) upon advancement of the inner member into the outer member and upon mutual engagement of the inner member and outer member threads:
i) the inner and outer trailing flanks are mutually engaged and substantially non parallel; and
ii) the inner and outer leading flanks, the inner root and outer crest surfaces, and the inner crest and outer root surfaces are substantially disengaged so as to form an anti-splay clearance therebetween, to thereby facilitate the outer member being drawn toward the inner member in response to the torquing the inner member within the opening.

27. In a medical implant having a pair of spaced arms and a closure for closing between the arms wherein the arms and closure include elements of a reverse angle thread with load flanks on the arms and closure for mating with each other and rotatably advancing the closure between the arms, the improvement wherein the load flanks are non parallel.

28. The implant according to claim 27 wherein the load flanksoutwardly diverge relative to one another away from the closure when originally joined.

29. The implant according to claim 27 wherein the load flanks outwardly converge relative to one another away from the closure when originally joined.

30. The implant according to claim 27 wherein each arm includes a break-off extension and the reverse angle thread extends into each extension.
